(19) United States
(12) Patent Application Publication
(10) Pub. No.: US 2012/0095538 A1
(43) Pub. Date: Apr. 19, 2012

(54) THERMAL WRAP ARRANGEMENT FOR APPLYING HEAT/COLD TO THE MANDIBULAR REGION OF THE HEAD

(76) Inventor: Rebecca A. Dow, Memphis, TN (US)

(21) Appl. No.: 13/346,670

(22) Filed: Jan. 9, 2012

Related U.S. Application Data
(63) Continuation-in-part of application No. 12/003,495, filed on Dec. 26, 2007.
Application No. 12/003,495 is a continuation-in-part of application No. 11/444,390, filed on Jun. 1, 2006, now abandoned.

(51) Int. Cl. A61F 7/00

(52) U.S. Cl. 607/109

(57) ABSTRACT

A thermal wrap having identical side portions, each having a pocket formed therein for receiving thermal material such as gel packs. The side portions are connected at the top by at least two independently attached straps, and at the bottom by a relatively wide chin strap to ensure that the pockets allow for even application of thermal energy to the mandibular region of the face. The pockets are sized to receive standard sized gel packs. In one embodiment the independently attached straps overlap a substantial portion of one of the side pockets to more precisely position and stabilize a gel pack over a treatment area.

### CROSS REFERENCE TO RELATED APPLICATIONS

This application is a continuation in part of application Ser. No. 12/003,495, filed on Dec. 26, 2007, which is a continuation in part of application Ser. No. 11/444,390, filed on Jun. 1, 2006.

### BACKGROUND OF THE INVENTION

1. Field of the Invention

The present invention concerns therapeutic thermal treatment devices. More particularly, the invention is directed to a thermal treatment wrap configured for application of heat/cold to various parts of a human head, particularly the lower mandibular region.

2. Description of the Prior Art

Thermal treatment wraps and the like are well known in the art. They typically consist of a web or sheet material which may be textile, elastic, or plastic; configured or configurable to cover a particular body part. They are typically conceived as one size fits all, or, in the alternative, limited to two or three sizes. While these arrangements are generally satisfactory, they can be ineffective when used to treat specific areas of the head and face. This is primarily because the human head, unlike other body parts, changes in both size and contour from one individual to the next, and allowances have to be made for the bulk of the hair, as well as particular hairstyles. To that end, the typical therapeutic thermal head wrap has adjustable or elasticated straps, and one or more pockets or voids for containing thermal material such as gel packs, ice, etc. The drawback with these types of arrangements is that it is difficult to maintain effective thermal contact with an area of the face to be treated. This problem is exacerbated by the use of gel packs which are dense, heavy, and not particularly conforming to contoured surfaces, the result being that the gel pack will either sag or pucker, and therefore be only marginally effective at a given treatment area. Prior art devices typically attempt to solve the problem by providing an elasticated material within which a gel pack is secured. The solution is only temporary at best, as the gel packs are heavy and wet and can deform the material irreversibly rendering it ineffective to accurately position the gel pack. Another problem with prior art devices is that the device will typically have a single large pocket for containing a gel pack, the pocket not conforming to any of the standard gel pack sizes. Another problem with existing therapeutic wraps and bandages is the use of elasticated material for much if not all of the wrap. This material, over time, stretches out of shape. The new shape does not allow for positioning the gel pack to affect the desired treatment area.

U.S. Pat. No. 5,837,004 issued to Lavore is typical of the head and face thermal treatment accessories. The device has opposing pockets for containing thermal material, the pockets connected at opposite ends by a single adjustable strap. The device is one size fits all. By contrast, the present invention is directed to a thermal compress arrangement having identical side portions connected at one end by a pair of elasticated straps, and at the other end by a chin strap.

U.S. Pat. No. 5,188,103 issued to Smith discloses a simple elastic band having a repositionable thermal pouch. The band will eventually stretch to a point that it cannot be held in place about the user's head at which point it must be replaced. The present invention contemplates a thermal treatment wrap designed for treatment of the mandibular region which has pockets sized in accordance with standard gel pack sizes. The wrap is available in a range of sizes, with the pocket size selected in accordance with both the standard gel pack sizes and the size of the user's head.

The present invention is directed to a thermal wrap having identical side portions, each having a pocket formed therein for receiving thermal material such as gel packs. The side portions are made of an expandable material and connected at the top by at least two independently attached non-elasticated straps, and at the bottom by a single, relatively wide non-elasticated chin strap to ensure that the pockets allow for even application of thermal energy to the mandibular region of the face. The pockets are sized to receive standard sized gel packs.

### SUMMARY OF THE INVENTION

It is a major object of the invention to provide an improved thermal wrap.

It is another object of the invention to provide an improved thermal wrap particularly adapted for use on a human head.

It is another object of the invention to provide an improved thermal wrap for treatment of the mandibular region of a human head.

It is another object of the invention to provide an improved, adjustable thermal wrap for treatment of the mandibular region of a human head.

It is a major object of the invention to provide an improved adjustable thermal wrap where the use of elastic material is limited to reduce stretching and permanent displacement of the thermal treatment material of the wrap.

It is yet another object of the invention to provide an improved thermal wrap with means for receiving standard sized thermal treatment packs.

Finally, it is a general goal of the invention to provide improved elements and arrangements thereof in an apparatus for the purposes described which is dependable and fully effective in accomplishing its intended purposes.

These and other objects of the present invention will become readily apparent upon further review of the following specification and drawings.

The present invention meets or exceeds all the above objects and goals. Upon further study of the specification and appended claims, further objects and advantages of this invention will become apparent to those skilled in the art.

### BRIEF DESCRIPTION OF THE DRAWINGS

Various other objects, features, and attendant advantages of the present invention will become more fully appreciated as the same becomes better understood when considered with the accompanying drawings, in which like reference characters designate the same or similar parts throughout the several views, and wherein:

FIG. 1 shows a front perspective view of the thermal wrap of the invention positioned on a user.

FIG. 2 shows a cross sectional side view of the thermal wrap.

FIG. 3 shows a cross sectional side view of the thermal wrap.

FIG. 4 shows a cross sectional plan view of the outer side of the thermal wrap with gel pack inserted.

FIG. 5 shows a cross sectional plan view of the inner side of the thermal wrap with gel packs inserted.

FIG. 6 shows a front perspective view of an alternative embodiment the thermal wrap of the invention positioned on a user.

FIG. 7 shows a cross sectional side view the alternative embodiment of the thermal wrap.

FIG. 8 shows a cross sectional side view of the alternative embodiment thermal wrap.

FIG. 9 shows a cross sectional plan view of the outer side of the alternative embodiment thermal wrap with gel pack inserted.

FIG. 10 shows a cross sectional plan view of the inner side of the alternative embodiment thermal wrap with gel pack inserted.

### DETAILED DESCRIPTION OF THE PREFERRED EMBODIMENTS

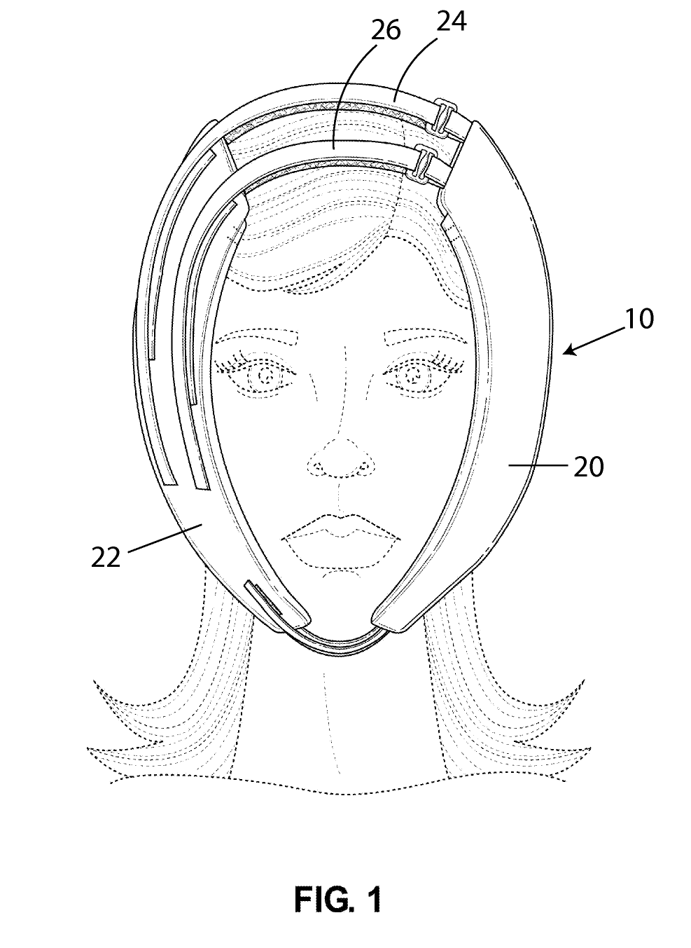

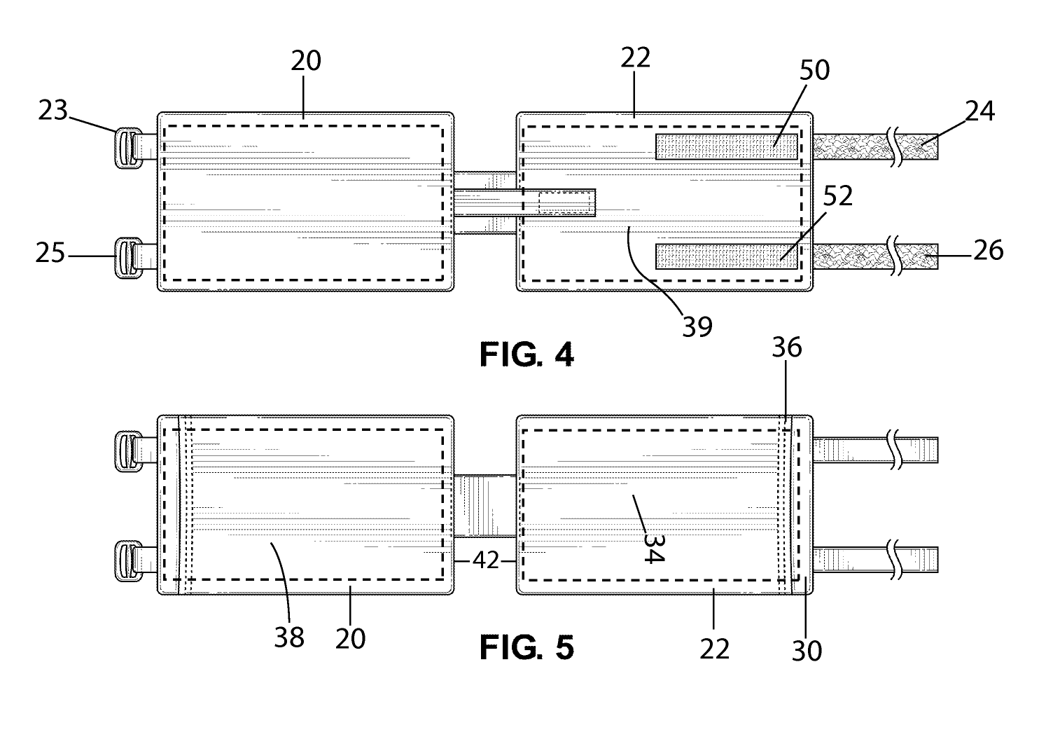

Referring now to FIGS. 1-5, the thermal wrap arrangement of the present invention, generally indicated by the numeral 10, is shown. It can be seen that the wrap 10 has a main body formed of two nearly identical side portions 20, 22, releasably connected by a pair of straps 24, 26 and corresponding pair of buckles 23, 25. Straps 24, 26, are evenly spaced about the center of opposing ends of the side portion 22, and are preferably connected thereto by stitching. Buckles 23, 25 are attached to a short length of straps 27, 29, which straps are attached by e.g. stitching to the top edge of pocket 20 to allow for adjustment as would be apparent to one of skill in the art. 27,29 elasticated pref Specifically, straps 24, 26 may be looped through one side of the respective buckle 23, 25 and adjusted as desired, and then secured by pressing the appropriate length of strap 24, 26 onto side portion 22 as will be described in more detail below.

It should be noted that while the side portions 20, 22 are shown as rectangular, they may have any elongated shape which can accommodate a side pocket which is sized to accept standard sized gel packs. The wrap 10 may be made available in incrementally increasing sizes starting from a small head (circumference) or child's head up to a maximum of about 28 inches. The size of the side portions 20, 22 are increased in accordance with the wrap 10 size increases, which are preferably about two inch increments, the side portions 20, 22 increased by about one inch for each wrap 10 size increase, with strap 24, 26 increasing about 2 inches. Of course the length of the straps may be increased or decreased to allow for additional adjustability. In a one size fits all embodiment, the side portions 20, 22 may be an intermediate size, while the straps 24, 26 are elongated to allow for adjustment from the smallest to the largest head size. The even spacing of the independently attached side straps 24, 26 help reduce twisting of the wrap 10 due to the thickness of the user's hair or the way in which the hair is arranged. It can be appreciated that more than two straps 24, 26 may be employed, especially for larger sizes of the wrap.

In a preferred embodiment, the wrap 10 is made from a polyester, spandex material, with the technical side of the material brushed or sueded to enhance user comfort. Also, the material may be treated with anti-microbial agents, and is knit to be quick drying. A material such as spandex, nylon, Lycra®, or a blend may be ideally used for this purpose. Also a woven material may be used. Any material used should be waterproofed or water resistant. The straps 24, 26 are preferably made from a woven hook and loop material such as Velcro®. Alternatively, the wrap 10 and straps 24, 26 may be made from any knit or woven material.

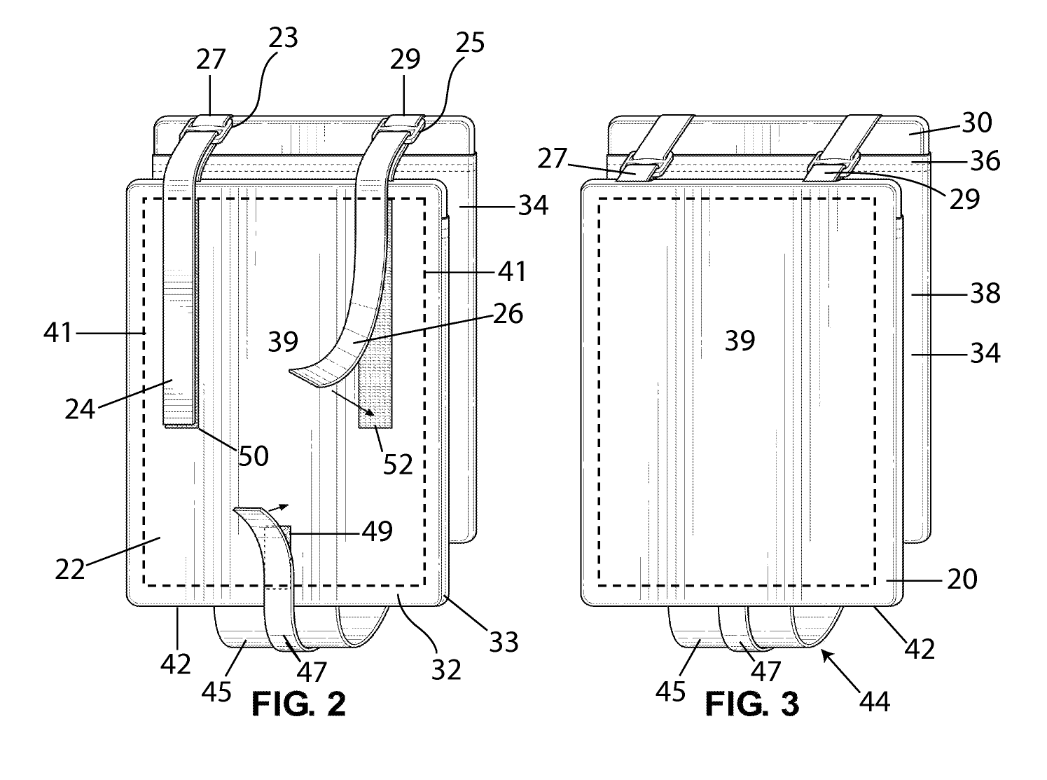

Referring specifically to FIGS. 2 and 3, it can be seen that each side portion 20, 22 has both inner and outer sheets 30, 32 which are attached to form a closed envelope 33, with a third sheet 34 unattached along its top edge 36 and thereby forming a pocket 38 into which a gel pack 39 may be inserted. The third sheet 34 is essentially a thermal transfer sheet as the gel pack 39 radiates thermal energy therethrough. The gel packs 39 may be hot or cold gel packs, or any type of thermal pack which is self contained and does not leak fluid, but preferably standard sized commercially available gel packs are used. Inner and outer sheets 30, 32 form an insulating barrier to limit the outward radiation of thermal energy from the gel pack 39. The attachment of sheets 30, 32 is preferably secured by stitching, with the top and bottom edges of the inner and outer sheets 30, 32 folded over to enhance durability. The top edge 36 of sheet 34 is preferably folded over and stitched and may be elasticated. The side pocket 38 is preferably wide enough so that the gel pack may be slid horizontally to adjust the region to which thermal energy is transferred.

The bottom edges 42 of the side pocket envelopes 33 are connected by stitching to a chin strap portion 44, which is formed of primary 45 and secondary 47 straps. The primary chin strap portion 45 is preferably made of spandex, nylon, or similar material as described above. The primary chin strap 45 is relatively wide to ensure that inner and outer sheets 30, 32 are stretched taut about the mandibular region of the user's face, and is preferably about half the width of the side pockets 38. Chin strap 45 is centrally disposed relative to straps 24 and 26. The secondary chin strap 47 is relatively thin, about a third to a half of the width of the primary chin strap 45, and centrally positioned relative to the position of straps 24, 26, and 45. Secondary chin strap is made from a Velcro® material. The sizing of the chin strap portion 44 relative to the width of the pockets 38 overcomes a tendency common in prior art thermal wraps where bunching or other fabric deformations due to a single, relatively thin strap lead to an uneven application of the thermal treatment. A chin strap similar in width to the pockets 38 (e.g., a wrap having the same width from end to end formed as a single constant width web of fabric) will cause deformation of the pocket material due to protrusion of the user's chin causing deformation of the web or sheet. Also, the positioning of the chin strap portion 44 allows for tensioning of the central part of the side portions 20, 22 in the lengthwise direction. Chin strap 47 is attached by stitching to the lower edge 42 of side portion 20 at one end, with the opposing end releasably attached to a lengthwise disposed strip 49 of hook and loop material.

Side portion 22, which may be designated the primary treatment or target side portion includes an opposing pair of elongated vertically or lengthwise disposed strips 50, 52 of hook and loop type fastening material positioned in spaced parallel relation on outer sheet 32. The strips 50, 52 extend about halfway, lengthwise, down the side portion 22 and are sized for releasable substantially overlapping engagement with straps 24, 26. The spacing of the strips 50, 52 corresponds to the width of a standard gel pack 39 such that when a gel pack is inserted into pocket 38 strips 50, 52 are positioned to apply pressure to lateral edge regions 41 of the gel pack via straps 24, 26. While the pocket 38 is sized to allow for some horizontal movement of the gel pack 39 once in the pocket, the width of the pocket 38 is limited so that some portion of the opposing edge regions 41 of the gel pack are directly beneath the portion of the straps 24, 26 which engage with strips 50, 52. The thickness of the gel pack 39 causes deformation of the inner and outer sheets 30, 32, which also creates lateral tension in the sheets to allow for better conformance of the gel pack 39 to the contours of the user's face on the target side 22 of the wrap 10. It should be noted that this is accomplished without the use of any laterally extending straps.

It can be seen that straps 24, 26 are elongated to extend nearly the entire length of side pocket 22, while not directly attached via strips 50, 52. This configuration allows for a wide range of adjustability of the wrap 10 while also compressing and deforming a gel pack 39 both laterally and vertically on the target side 22 of the wrap due to the action of straps 24, 26, and the chin strap portion 44, respectively, as described above.

In use, once the user has selected a wrap 10 size appropriate for his/her head size, the appropriate sized gel pack 39 is inserted. The wrap 10 is then positioned about the user's head. Straps 24, 26 are then engaged with hook and loop strips 50, 52 to secure the pocket 22 and enclosed gel pack 39 onto the target area of the user's face as seen in FIG. 1 The straps 24, 26 may be re-adjusted to closely conform the wrap 10 about the user's head and to apply the thermal energy from the gel pack more effectively. Finally, strap 47 may be attached via strip 49 to side portion 22, if desired, to create more tension in the immediate lower mandibular region when the wrap 10 is applied thereto. This tension can be selectively reduced by releasing the strap 47, without having to loosen straps 24, 26. It can be appreciated that the configuration of the wrap 10 allows primarily for treatment of the lower mandibular region of a human head but due to the interaction of straps 24, 26 and chin strap 44, the wrap 10 may be adjusted to treat practically any area of the head or neck. In the event that the user needs to treat both sides of the head side portion 20 also contains a pocket 38 as described above, but without the additional conformability of the target side 22.

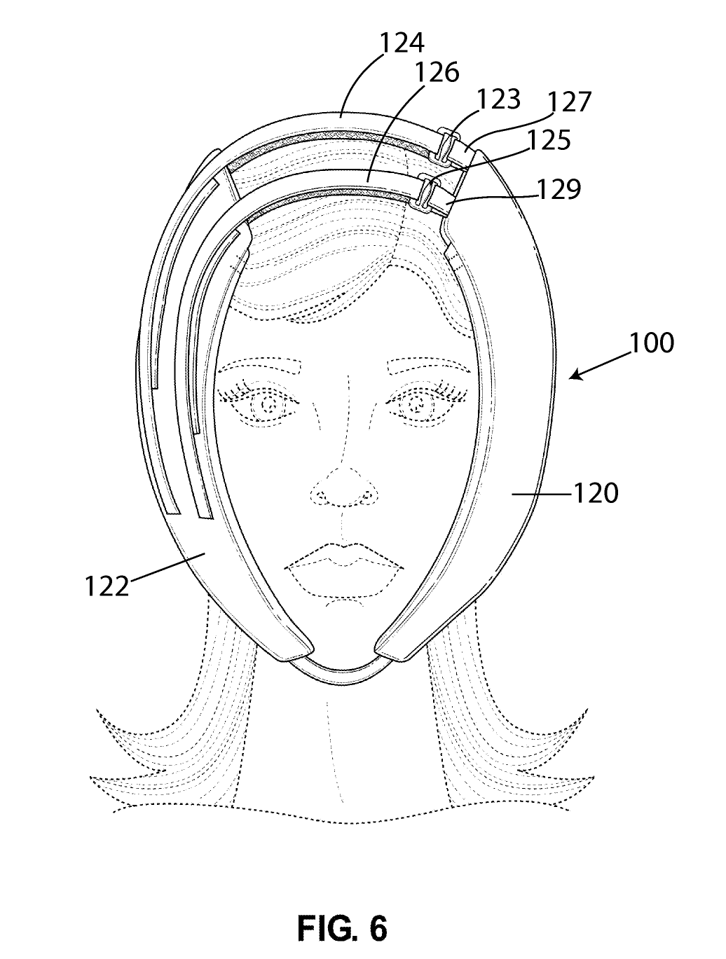

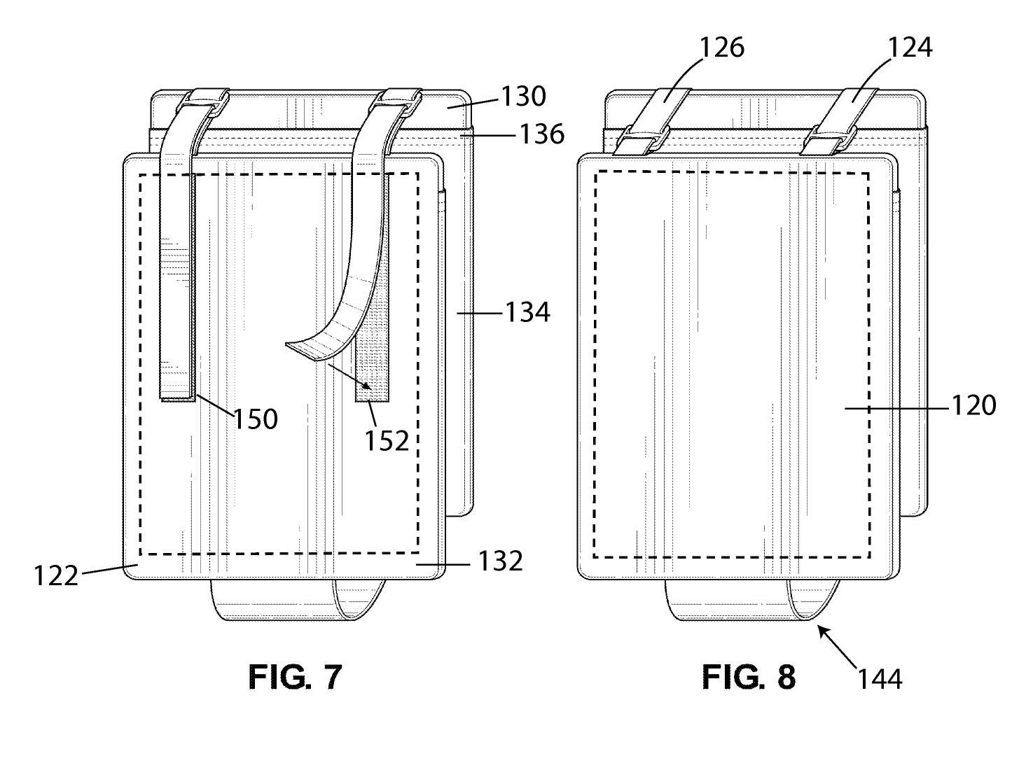

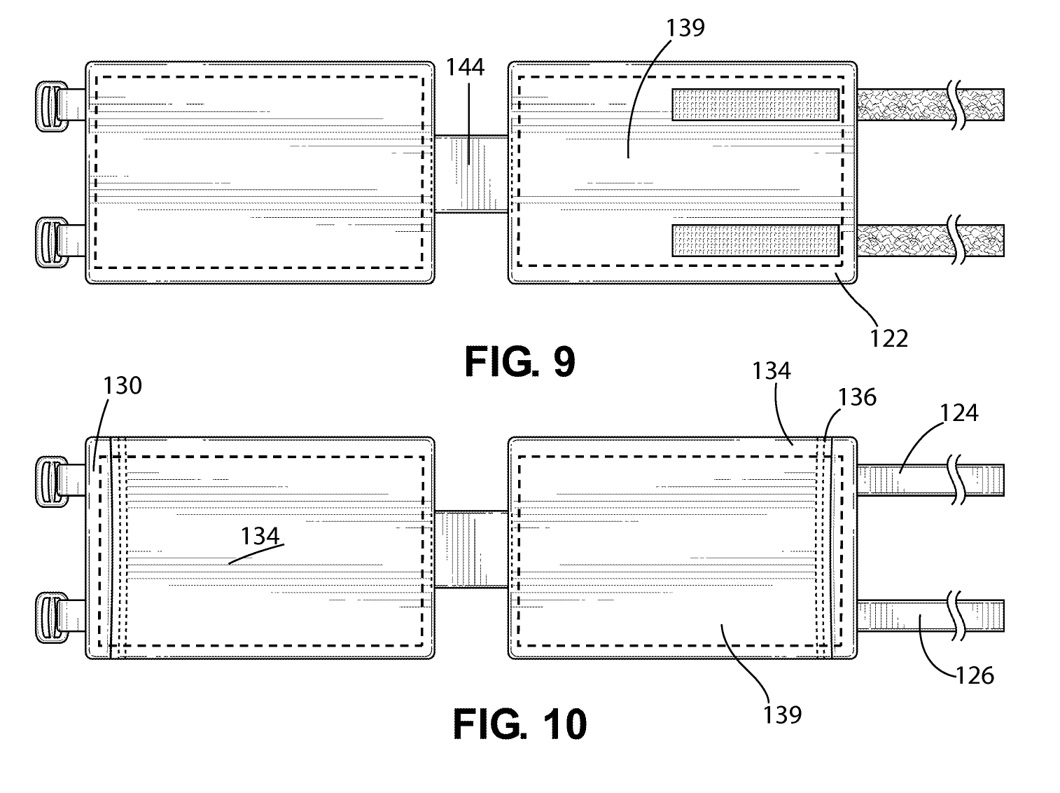

FIGS. 6-10 show an alternative embodiment of the wrap, generally designated by the numeral 100. The wrap 100 is made from the same materials as described above. The wrap 100 has a main body formed of two nearly identical side portions 120, 122, releasably connected by a pair of elasticated straps 124, 126 and corresponding pair of buckles 123, 125. Straps 124, 126, are evenly spaced about the center of opposing ends of the side portion 22, and are preferably connected thereto by stitching. Buckles 123, 125 are attached to a short length of straps 127, 129, which straps are attached by e.g. stitching to the top edge of pocket 120 to allow for adjustment as would be apparent to one of skill in the art. Specifically, straps 124, 126 may be looped through one side of the respective buckle 123, 125 adjusted as desired, and then secured by pressing the appropriate length of strap 124, 126 onto side portion 122 via strips 150 and 152.

As with the previous embodiment, each side portion 120, 122 has both inner and outer sheets 130, 132 which are attached to form a closed envelope 133, with a third sheet 134 unattached along its top edge 136 and thereby forming a pocket 138 into which a gel pack 139 may be inserted. The third sheet 134 is essentially a thermal transfer sheet as the gel pack 139 radiates thermal energy therethrough. Inner and outer sheets 130, 132 thus form an insulating barrier to limit the outward radiation of thermal energy from the gel pack 139. The attachment of sheets 130, 132 is preferably secured by stitching, with the top and bottom edges of the inner and outer sheets 130, 132 folded over to enhance durability. The top edge 136 of sheet 134 is preferably folded over and stitched and may be elasticated. The side pocket 138 is wide enough so that the gel pack may be slid horizontally to adjust the region to which thermal energy is transferred. Chin strap 144 is attached to lower edges of side portion 120, 122 by stitching. The wrap 100 is applied to a user's head as described above, with the exception that the chin strap 144 is not adjustable.

From the foregoing description, one skilled in the art can easily ascertain the essential characteristics of this invention and, without departing from the spirit and scope thereof, can make various changes and modifications of the invention to adapt it to various usages and conditions.

It is to be understood that the present invention is not limited to the sole embodiment described above, but encompasses any and all embodiments within the scope of the following claims:

I claim:

1. A thermal wrap for positioning about a user's head comprising:
said wrap having a pair of opposing side portions, each of said side portions having inner and outer sheets, said side portions selectively connected by at least a pair of horizontally spaced connecting straps, the connecting straps connecting top ends of the side portions;
said wrap including a chin strap portion connected to lower edges of said side portions;
each of said side portions including a pocket for receiving a pack of thermal treatment material;
one of said side portions having a pair of co-extensive strips of hook and loop type fastening material positioned on the outer sheet in overlapping relation to said pocket to form a primary treatment side of said wrap;
said straps engageable with said strips of hook and loop material to secure the wrap in position about the user's head and secure said primary treatment side in position proximate a target position on said user's face.

2. The wrap of claim 1 wherein one side of said inner sheets is brushed.

3. The wrap of claim of claim 1 wherein said chin strap portion is about one half the width of said side pockets.

4. The wrap of claim 1 wherein the width of said pocket allows for lateral repositioning of said gel pack.

5. The wrap of claim 1 wherein said chin strap portion includes a permanently attached strap and a releasably attached strap.

6. A thermal wrap for positioning about a user's head comprising:
said wrap having a pair of opposing side portions, each of said side portions having inner and outer sheets, said side portions selectively connected by at least a pair of horizontally spaced connecting straps, the connecting straps connecting top ends of the side portions;
said wrap including a chin strap portion connected to lower edges of said side portions;
one of said side portions having a pair of co-extensive strips of hook and loop type fastening material positioned on the outer sheet to form a primary treatment side of said wrap;
each of said side portions including a pocket for receiving a pack of thermal treatment material;
said connecting straps engageable with said strips of hook and loop material to secure the wrap in position about the user's head and apply lateral tension to said inner and outer sheets of said primary treatment side of said wrap, said chin strap portion centrally positioned to apply lengthwise tension to said inner and outer sheets of said primary treatment side of said wrap.

7. The wrap of claim of claim 6 wherein said chin strap portion is about one half the width of said side pockets.

8. The wrap of claim 6 wherein the width of said pocket allows for lateral repositioning of said gel pack.

9. The wrap of claim 6 wherein said chin strap portion includes a permanently attached strap and a releasably attached strap.
